(19) United States
(12) Patent Application Publication
(10) Pub. No.: US 2014/0199764 A1
(43) Pub. Date: Jul. 17, 2014

(54) MICROFLUIDIC MODULE AND USES THEREOF

(71) Applicants: Karel Domansky, Charlestown, MA (US); Daniel C. Leslie, Brookline, MA (US); Geraldine A. Hamilton, Cambridge, MA (US); Anthony Bahinski, Wilmington, DE (US); Donald E. Ingber, Boston, MA (US)

(72) Inventors: Karel Domansky, Charlestown, MA (US); Daniel C. Leslie, Brookline, MA (US); Geraldine A. Hamilton, Cambridge, MA (US); Anthony Bahinski, Wilmington, DE (US); Donald E. Ingber, Boston, MA (US)

(73) Assignee: PRESIDENT AND FELLOWS OF HARVARD COLLEGE, Cambridge, MA (US)

(21) Appl. No.: 14/116,481

(22) PCT Filed: May 8, 2012
(86) PCT No.: PCT/US2012/036920

§ 371 Date: Feb. 14, 2014

Related U.S. Application Data
(60) Provisional application No. 61/483,990, filed on May 9, 2011.
(60) Provisional application No. 61/541,821, filed on Sep. 30, 2011.

(51) Int. Cl. C12N 5/071; B32B 37/18

(52) U.S. Cl. CPC C12N 5/0602; B32B 37/18
USPC 435/396; 435/289.1; 156/242

(57) ABSTRACT

Described herein are microfluidic modules and methods for making the same, wherein the microfluidic modules include a substrate comprising at least one ether-based, aliphatic polyurethane, and at least one fluidic element disposed therein. The ether-based aliphatic polyurethane can be either the substrate of the microfluidic modules or a coating of another substrate material, such that at least a portion of the ether-based, aliphatic polyurethane is in fluid communication. In one embodiment, the ether-based, aliphatic polyurethane includes dicyclohexylmethane-4,4′-diisocyanate. As the ether-based aliphatic polyurethane can decrease absorption of molecules, e.g., hydrophobic molecules, in such microfluidic modules, the microfluidic modules described herein can be used in various applications such as drug screening and fluorescent microscopy.

### RELATED APPLICATIONS

This application claims benefit under 35 U.S.C. §119(e) of the U.S. Provisional Application No. 61/483,990 filed May 9, 2011, and U.S. Provisional Application No. 61/541,821 filed Sep. 30, 2011, the content of both of which is incorporated herein by reference in its entirety.

### GOVERNMENT SUPPORT

This invention was made with support from the federal government under Grant No. 1U01NS073474-01, awarded by the National Institutes of Health/National Institute of Neurological Disorders and Stroke. The U.S. Government has certain rights in the invention.

### TECHNICAL FIELD

The present disclosure relates generally to improvement of microfluidic devices.

### BACKGROUND

Poly(dimethylsiloxane) (PDMS) has been widely used in fabrication of microfluidic devices by virtues of its simple fabrication process and material attributes, such as optical transparency, gas permeability and flexibility. Cured PDMS is a crosslinked polymer of hydrophobic dimethylsiloxane oligomers. Thus, small hydrophobic molecules such as drugs, fluorescent dyes, or cell signaling molecules are strongly absorbed in PDMS microfluidic devices, resulting in time-dependent solution concentrations, cross-contamination, lower detection sensitivity, and/or higher background autofluorescence. Partitioning of molecules into the bulk is in part behind the slow industrial acceptance of PDMS microfluidic devices. This issue could severely limit the utility of microfluidic devices, specifically in drug screening applications.

While there are clear and flexible materials such as perfluoropolyethers that make inroads into the fabrication of microfluidic devices, they do not prevent absorption of small hydrophobic molecules. See for example Devaraju and Unger (Lab Chip, 2011, 11: 1962-1967). Parlyene-coated PMDS devices have also been investigated, but parylene films are stiffer and their deposition requires the use of specialized instruments.

Accordingly, there is still a strong need for improvement of a microfluidic device, in particular, to overcome the absorption of hydrophobic molecules therein.

### SUMMARY

Described herein is a microfluidic module comprising a substrate and at least one fluidic element disposed therein, wherein the substrate comprises at least one ether-based, aliphatic polyurethane, and wherein at least a portion of the at least one ether-based, aliphatic polyurethane is in fluid communication. The at least one ether-based, aliphatic polyurethane is optically clear, decreases absorption of molecules, e.g., hydrophobic molecules, and allows for cell culture. In one embodiment, the at least one ether-based, aliphatic polyurethane comprises dicyclohexylmethane-4,4′-diisocyanate, its derivatives and/or its isomers thereof. Such microfluidic modules can be used for various applications, for example, assays involving hydrophobic molecules, such as drug screening, cell signaling study, and fluorescent microscopy.

Another aspect described herein is a method of making a microfluidic module from at least one ether-based, aliphatic polyurethane, wherein at least a portion of the at least one ether-based, aliphatic polyurethane is in fluid communication. In one embodiment, the at least one ether-based, aliphatic polyurethane comprises dicyclohexylmethane-4,4′-diisocyanate, its derivatives and/or its isomers thereof. In some embodiments, the microfluidic module can be formed by replica molding. In some embodiments, the microfluidic module can be formed by micromachining. In some embodiments, the microfluidic module can be formed by solid-object printing.

In additional aspect, described herein is ether-based, aliphatic polyurethane for use in inhibiting absorption of molecules, e.g., hydrophobic molecules, in a microfluidic module, wherein at least a portion of the ether-based, aliphatic polyurethane is in fluid communication. In one embodiment, the ether-based, aliphatic polyurethane comprises dicyclohexylmethane-4,4′-diisocyanate, its derivatives and/or its isomers thereof.

### BRIEF DESCRIPTION OF THE DRAWINGS

FIGS. 1A-1D show absorption of dyes into ether-based, aliphatic polyurethane and PDMS. Discs were soaked in dye solutions for 48 hours, rinsed with water, and air-dried. FIG. 1A shows that a 2-mm thick slice was sectioned from each disc. The discs were laid flat and imaged from a cut side. FIG. 1B shows absorption of Nile red solution into PDMS, but not ether-based, aliphatic polyurethane. FIG. 1C shows absorption of rhodamine B solution into PDMS, but not ether-based, aliphatic polyurethane. FIG. 1D shows little or no absorption of FITC solution into PDMS or ether-based, aliphatic polyurethane.

FIG. 2 is a photograph of optically clear and flexible 3-channel microfluidic devices fabricated from PDMS (left) and ether-based, aliphatic polyurethane (right). The devices were corona-bonded to 22×50-mm microscope cover-slips. The main channel in each device is 400 μm and the side channels are 200 μm. All channels are 70 μm deep. The PDMS device was cast directly from a silanized silicon wafer with SU-8 resist features. Because of a stronger adhesion of polyurethane to silanized silicon masters, the polyurethane device was cast from a silicone mold replicated from the original silicon master.

FIG. 3 is a photograph of microchannels of the corona-bonded ether-based, aliphatic polyurethane microfluidic device shown in FIG. 2 invention filled with food-colored aqueous solutions.

FIG. 4 shows HUVEC cells cultured on fibronectin-coated ether-based, aliphatic polyurethane discs that were inserted into a 48-well tissue culture plate. Image was taken 3 hours after seeding.

FIG. 5 shows bonding under different surface pre-treatment conditions.

FIG. 6 shows the effect of UV ozone sterilization of 1552-2 GS polyurethane on water contact angle. Samples were positioned 5 mm from the UV lamp.

FIG. 7 shows the effect of UV-ozone treatment of 1552-2 GS polyurethane on human umbilical vein endothelial cell (HUVEC) adhesion. Following the UVO treatment, polyurethane was coated with fibronectin by immersion in a 20 μg/ml fibronectin solution in 50 mM carbonate buffer at pH 9.3 and 4° C. for 24 hours. Images were taken on day 1 and day 4.

FIG. 8 shows the effect of leachables on cell viability.

FIG. 9 shows the hydrophobic recovery of 1552-2 GS polyurethane treated with air plasma for 30 s, corona discharge for 2 minutes, and UV ozone for 10 minutes.

FIG. 10 shows the optical transmission of cast 1552-2 GS polyurethane. The samples were approximately 2 mm in thickness. Two polyurethane and two PDMS samples are plotted. The measurements were performed with Cary 300 spectrophotometer at room temperature.

FIGS. 11A-11C shows the performance of 1552-2 GS polyurethane subjected to cyclical load testing on Instron 5544 tensometer. As shown in FIG. 11A, the samples were tested at elongation ranging from 0% to 10% with 8-second cycle period. FIG. 11B shows the resistive force at 10% elongation as a function of number of cycles. FIG. 11C shows the resistive force normalized by the initial force.

FIG. 12 shows the stress strain curve of polyurethane and PDMS

FIG. 13 shows one embodiment of a multi-step molding process for fabricating a device.

FIGS. 14A-14C show the casting of elastomeric GS polyurethane microfluidic devices according to an embodiment of a multi-step molding process for fabricating a device.

FIG. 14A shows the PDMS replicas prepared by casting on SU-8 on silicon masters. FIG. 14B shows the hard 310 polyurethane molds prepared by casting on PDMS replicas. FIG. 14C shows elastomeric GS polyurethane parts after they are peeled from the silanized hard 310 polyurethane molds.

FIGS. 15A and 15B show fabrication of porous polyurethane membrane. FIG. 15A shows the patterned elastomeric GS polyurethane on PMDS “handle” slabs. The pores are approximately 7 μm in diameter. FIG. 15B shows a piece of freestanding GS polyurethane membrane after it is peeled off from the PDMS slab. The membrane is approximately 50 μm in thickness.

### DETAILED DESCRIPTION OF EXEMPLARY EMBODIMENTS

Strong absorption of hydrophobic molecules renders poly(dimethylsiloxane) (PDMS)-based microfluidic devices or modules undesirable in various applications ranging from studying cell response, to small-molecule screening, to fluorescent microscopy. As such, it is important to improve the performance of microfluidic devices for use involving hydrophobic molecules. In accordance with the invention, microfluidic devices fabricated from ether-based, aliphatic polyurethane, for example, comprising dicyclohexylmethane-4,4′-diisocyanate, provides for decreased absorption of molecules, e.g., hydrophobic molecules. The ether-based aliphatic polyurethane is optically clear, and biocompatible, thus allowing microscopy and cell culture.

Accordingly, in one aspect, provided herein is a microfluidic module comprising a substrate and at least one fluidic element disposed therein, wherein the substrate comprises at least one ether-based, aliphatic polyurethane, and wherein at least a portion of the at least one ether-based, aliphatic polyurethane is in fluid communication. Another aspect is a method of making a microfluidic module, comprising forming a microfluidic module from at least one ether-based, aliphatic polyurethane, wherein the microfluidic module comprises a substrate and at least one fluidic element disposed therein, and wherein at least a portion of the at least one ether-based, aliphatic polyurethane is in fluid communication.

### Microfluidic Modules

Microfluidic modules are microscale structures widely used in chemistry and biological applications. Different microfluidic modules have been designed and developed in the art for various applications, e.g., measuring molecular diffusion coefficients (Kamholz, A. E., Weigl, B. H., Finlayson, B. A. & Yager, P. Quantitative analysis of molecular interaction in a microfluidic channel: The T-sensor. Analytical Chemistry, 1999, 71: 5340-5347 and Kamholz, A. E., Schilling, E. A. & Yager, P. Optical measurement of transverse molecular diffusion in a microchannel. Biophysical Journal, 2001, 80: 1967-1972), fluid viscosity (Galambos, P. Ph.D. Thesis, Mechanical Engineering. University of Washington, Seattle (1998), pH (Weigl, B. H. & Yager, P. Silicon-microfabricated diffusion-based optical chemical sensor. Sensors and Actuators B-Chemical, 1997, 39: 452-457 and Macounova, K., Cabrera, C. R., Holl, M. R. & Yager, P. Generation of natural pH gradients in microfluidic channels for use in isoelectric focusing. Analytical Chemistry, 2000, 72: 3745-3751), chemical binding coefficients (Kamholz, A. E., Weigl, B. H., Finlayson, B. A. & Yager, P. Quantitative analysis of molecular interaction in a microfluidic channel: The T-sensor. Analytical Chemistry, 1999, 71: 5340-5347) and enzyme reaction kinetics (Hadd, A. G., Raymond, D. E., Halliwell, J. W., Jacobson, S. C. & Ramsey, J. M. Microchip device for performing enzyme assays. Analytical Chemistry, 1997, 69: 3407-3412; Duffy, D. C., Gillis, H. L., Lin, J., Sheppard, N. F. & Kellogg, G. J. Microfabricated centrifugal microfluidic systems: Characterization and multiple enzymatic assays. Analytical Chemistry, 1999, 71: 4669-4678; and Hadd, A. G., Jacobson, S. C. & Ramsey, J. M. Microfluidic assays of acetylcholinesterase inhibitors. Analytical Chemistry, 1999, 71: 5206-5212). Other applications for microfluidic modules include capillary electrophoresis (Kameoka, J., Craighead, H. G., Zhang, H. W. & Henion, J. A polymeric microfluidic chip for CE/MS determination of small molecules. Analytical Chemistry, 2001, 73: 1935-1941), isoelectric focusing (Macounova, K., Cabrera, C. R., Holl, M. R. & Yager, P. Generation of natural pH gradients in microfluidic channels for use in isoelectric focusing. Analytical Chemistry, 2000, 72: 3745-3751; Xu, J., Lee, C. S. & Locascio, L. E. Isoelectric focusing of green fluorescence proteins in plastic microfluid channels. Abstracts of Papers of the American Chemical Society, 2000, 219: 9-ANYL; and Macounova, K., Cabrera, C. R. & Yager, P. Concentration and separation of proteins in microfluidic channels on the basis of transverse IEF. Analytical Chemistry, 2001, 73: 1627-163), immunoassays (Hatch, A. et al. A rapid diffusion immunoassay in a T-sensor. Nature Biotechnology, 2001, 19: 461-465; Eteshola, E. & Leckband, D. Development and characterization of an ELISA assay in PDMS microfluidic channels. Sensors and Actuators B-Chemical, 2001, 72: 129-133; Cheng, S. B. et al. Development of a multichannel microfluidic analysis system employing affinity capillary electrophoresis for immunoassay. Analytical Chemistry, 2001, 73: 1472-1479; and Yang, T. L., Jung, S. Y., Mao, H. B. & Cremer, P. S. Fabrication of phospholipid bilayer-coated microchannels for on-chip immunoassays. Analytical Chemistry, 2001, 73: 165-169), flow cytometry (Sohn, L. L. et al. Capacitance cytometry: Measuring biological cells one by one. Proceedings of the National Academy of Sciences of the United States of America, 2000, 97: 10687-10690), sample injection of proteins for analysis via mass spectrometry (Figeys, D., Gygi, S. P., McKinnon, G. & Aebersold, R. An integrated microfluidics tandem mass spectrometry system for automated protein analysis. Analytical Chemistry, 1998, 70: 3728-3734; Jiang, Y., Wang, P. C., Locascio, L. E. & Lee, C. S. Integrated plastic microfluidic devices with ESI-MS for drug screening and residue analysis. Analytical Chemistry, 2001, 73: 2048-2053; and Gao, J., Xu, J. D., Locascio, L. E. & Lee, C. S. Integrated microfluidic system enabling protein digestion, peptide separation, and protein identification. Analytical Chemistry, 2001, 73: 2648-2655), PCR amplification (Belgrader, P., Okuzumi, M., Pourahmadi, F., Borkholder, D. A. & Northrup, M. A. A microfluidic cartridge to prepare spores for PCR analysis. Biosensors & Bioelectronics, 2000, 14: 849-852; Khandurina, J. et al. Integrated system for rapid PCR-based DNA analysis in microfluidic devices. Analytical Chemistry, 2000, 72: 2995-3000; Lagally, E. T., Medintz, I. & Mathies, R. A. Single-molecule DNA amplification and analysis in an integrated microfluidic device. Analytical Chemistry, 2001, 73: 565-570), DNA analysis (Buchholz, B. A. et al. Microchannel DNA sequencing matrices with a thermally controlled “viscosity switch”. Analytical Chemistry, 2001, 73: 157-164; Fan, Z. H. et al. Dynamic DNA hybridization on a chip using paramagnetic beads. Analytical Chemistry, 1999, 71: 4851-4859; Koutny, L. et al. Eight hundred base sequencing in a microfabricated electrophoretic device. Analytical Chemistry, 2000, 72: 3388-3391; and Lee, G. B., Chen, S. H., Huang, G. R., Sung, W. C. & Lin, Y. H. Microfabricated plastic chips by hot embossing methods and their applications for DNA separation and detection. Sensors and Actuators B-Chemical, 2001, 75: 142-148 (2001), cell manipulation (Glasgow, I. K. et al. Handling individual mammalian embryos using microfluidics. Ieee Transactions On Biomedical Engineering, 2001, 48: 570-578), cell separation (Yang, J., Huang, Y., Wang, X. B., Becker, F. F. & Gascoyne, P. R. C. Cell separation on microfabricated electrodes using dielectrophoretic/gravitational field flow fractionation. Analytical Chemistry, 1999, 71: 911-918), cell patterning (Chiu, D. T. et al. Patterned deposition of cells and proteins onto surfaces by using three-dimensional microfluidic systems. Proceedings of the National Academy of Sciences of the United States of America, 2000, 97: 2408-2413 and Folch, A., Jo, B. H., Hurtado, O., Beebe, D. J. & Toner, M. Microfabricated elastomeric stencils for micropatterning cell cultures. Journal of Biomedical Materials Research, 2000, 52: 346-353), chemical gradient formation (Dertinger, S. K. W., Chiu, D. T., Jeon, N. L. & Whitesides, G. M. Generation of gradients having complex shapes using microfluidic networks. Analytical Chemistry, 2001, 73: 1240-1246 and Jeon, N. L. et al. Generation of solution and surface gradients using microfluidic systems. Langmuir, 2000, 16: 8311-8316), and clinical diagnostics (Weigl, B. H. & Yager, P. Microfluidics—Microfluidic diffusion-based separation and detection. Science, 1999, 283: 346-347 and Cunningham, D. D. Fluidics and sample handling in clinical chemical analysis. Analytica Chimica Acta, 2001, 429: 1-181).

In some embodiments, the microfluidic modules can be used as synthesis micro-reactors, e.g., for producing any compounds of interests (such as molecules, particles, and emulsions) from starting reactants introduced into the microfluidic modules or devices. In some embodiments, the microfluidic modules can be used for microanalysis, for example, to detect specific compounds, and/or to detect their content, in specimens of a variety of sources, e.g., in biological fluids. In some embodiments, the microfluidic modules can be designed to function as heat exchangers, filters, mixers, extractors, separators (for example those operating by electrophoresis), devices for generating droplets of a given size or solid particles, or as devices for carrying out particular operations (e.g., cell lysis, DNA amplification). In some embodiments, the microfluidic modules can be adapted to use as cell culture platforms or bioreactors. In such embodiments, the microfluidic module can comprise at least one cell. In some embodiments, the microfluidic modules, also referred as “organ-on-a-chips,” can be designed to mimic physiological functions of an organ or a tissue, for example, but not limited to, the ones disclosed in the PCT patent applications WO 2010/009307, and PCT/US2010/021195; and U.S. Provisional Patent Applications 61/477,540, 61/449,925, and 61/449,925.

In some embodiments, a microfluidic module comprises a substrate and at least one fluidic element disposed therein. The number of fluidic elements in a microfluidic module can vary depending on the design and/or application of the microfluidic module. One of skill in the art will be able to design and determine optimum number of fluidic elements required to achieve a certain application. In some embodiments, the microfluidic module can be a stand-alone microfluidic device. In some embodiments, the microfluidic module can be one component or unit of a device or a system.

The term “substrate” as used herein includes a support material in which at least one fluidic element is disposed. In some embodiments, the substrate can comprise any material such as glass, co-polymer, polymer or any combinations thereof. Exemplary polymers include, but are not limited to, polyurethanes, rubber, molded plastic, polymethylmethacrylate (PMMA), polycarbonate, polytetrafluoroethylene (TEFLON™), polyvinylchloride (PVC), polydimethylsiloxane (PDMS), and polysulfone. In some embodiments, the substrate comprises polyurethanes. In one embodiment, the substrate comprises at least one ether-based, aliphatic polyurethane, wherein at least a portion of the ether-based aliphatic polyurethane is in fluid communication. In such embodiments, the ether-based aliphatic polyurethane can be either the substrate material in contact with a fluid flowing through a fluidic element, or a coating of the substrate material, wherein the coating is in contact with a fluid flowing through a fluidic element. In accordance with the invention, such ether-based aliphatic polyurethane polymer provides advantages of decreasing or inhibiting absorption of molecules, e.g., hydrophobic molecules, in addition to optical transparency, flexibility and biocompatibility. In various embodiments, any substrate material other than ether-based aliphatic polyurethane (e.g., PDMS) can be excluded from fluid communication. For example, those substrate materials can be coated or layered with at least one ether-based, aliphatic polyurethane. In one embodiment, PDMS is excluded from fluid communication. In such embodiment, the PDMS can be coated or layered with at least one ether-based, aliphatic polyurethane.

As used herein, the term “fluidic element” is used in reference to a microfluidic element capable of containing and/or transporting a fluid regardless of the cross-sectional shape. By way of example, the fluidic element can have a cross-section with a shape of approximately square, rectangle, trapezoid, oval or circle. The fluid can be stored in or flow through at least one fluidic element depending upon various types of applications.

In some embodiments, the fluidic element can be a microchannel. The term “microchannel” as used herein refers to a channel formed in a microfluidic module or device having cross-sectional dimensions in the range between about 0.1 μm and about 500 μm, between about 0.5 μm and about 250 μm, or between about 5 μm and about 100 μm.

In some embodiments, the fluidic element can be a microwell. A “microwell” refers to a micro-scale chamber able to accommodate a fluid. A microwell is generally defined by a curved surface, which is concave. In some embodiments, the microwell has a dimension in the range between about 0.1 μm and about 2000 μm, between about 100 μm and about 1000 μm, or between about 250 μm and about 500 μm.

In some embodiments, at least one fluidic element can further be coated with one or more cell adhesion molecules, e.g., to promote cell attachment to a surface of the at least one fluidic element. Exemplary cell adhesion molecules include, but are not limited to, fibronectin, collagen, gelatin, laminin, vitronectin, fibrin, and any combinations thereof.

In some embodiments, the microfluidic module can further comprises at least one inlet and/or at least one outlet, which are connected via one or more fluidic elements. The inlets and/or outlets of the microfluidic module can be connected to a pump, e.g., with a tubing.

The methods used in fabrication of any embodiments of the microfluidic module described herein can vary with the materials used, and include soft lithography methods, microassembly, bulk micromachining methods, surface micro-machining methods, standard lithographic methods, wet etching, reactive ion etching, plasma etching, stereolithography and laser chemical three-dimensional writing methods, solid-object printing, machining, modular assembly methods, replica molding methods, injection molding methods, hot molding methods, laser ablation methods, combinations of methods, and other methods known in the art. A variety of exemplary fabrication methods are described in Fiorini and Chiu, 2005, “Disposable microfluidic devices: fabrication, function, andapplication” Biotechniques 38:429-446; Beebe et al., 2000, “Microfluidic tectonics: a comprehensive construction platform for microfluidic systems.” Proc. Natl. Acad. Sci. USA 97:13488-13493; Rossier et al., 2002, “Plasma etched polymer microelectrochemical systems” Lab Chip 2:145-150; Becker et al., 2002, “Polymer microfluidic devices” Talanta 56:267-287; Becker et al., 2000, “Polymer microfabrication methods for microfluidic analytical applications” Electrophoresis 21:12-26; U.S. Pat. No. 6,767,706 B2, e.g., Section 6.8 “Microfabrication of a Silicon Device”; McDonald et al., 2002, “Poly(dimethylsiloxane) as a material for fabricating microfluidic devices” Accounts of Chemical Research 35: 491-499. Piccin et al., 2007, “Polyurethane from biosource as a new material for fabrication of microfluidic devices by rapid prototyping” Journal of Chromatography A 1173: 151-158. Each of these references are incorporated herein by reference in their entirety.

In some embodiments, a microfluidic module described herein can be formed by replica molding, for example, in which a replica comprising at least one ether-based, aliphatic polyurethane conforms to the shape of a master or a mold and replicates the features of the master or the mold. In some embodiments, the replica can be further sealed to a surface to enclose at least one fluidic element.

In some embodiments, a microfluidic module described herein can be formed by machining or micromachining. The term “micromachining” as used herein can encompass bulk micromachining or surface micromachining as recognized in the art. In one embodiment, bulk micromachining defines microstructures such as fluidic elements by selectively etching inside a substrate. In one embodiment, surface micromachining creates microstructures such as fluidic elements on top of a substrate.

In some embodiments, a microfluidic module described herein can be formed by solid-object printing. In some embodiments, the solid-object printing can take a three-dimensional (3D) computer-aided design file to make a series of cross-sectional slices. Each slice can then be printed on top of one another to create the 3D solid object.

In additional embodiments, a microfluidic module described herein can further comprise at least one additional component, for example, without limitations, to control fluid flow, to apply a pressure, to modulate light or provide an optical effect, to modulate and/or provide electricity, and/or to allow filtration of a fluid. Non-limiting examples of additional components that can be integrated with a microfluidic module include glass capillaries, silicone tubing, optical fibers, electronic devices, membranes, valves, pumps, and any combinations thereof.

### Ether-Based, Aliphatic Polyurethane

Polyurethanes are a very broad class of polymers that have been used in many applications including the biomedical industry. Polyurethanes are any polymers consisting of a chain of monomers joined by urethane links. Polyurethanes are generally formed by reacting monomers containing at least two isocyanate functional groups (e.g., a diisocyanate containing two —NCO groups) with other monomers containing at least two hydroxyl (alcohol) groups (e.g., a polyol containing at least two —OH groups). The isocyanate and polyol monomers during the reaction can be long, short, aliphatic or aromatic, producing polyurethanes with diverse physical and/or chemical properties, such as optical clarity, color, flexibility, hydrophilicity, biocompatibility, and/or different chemical interactions.

Recently, thin polyurethane films have been integrated into PDMS or rigid polymer devices. See, for example, Moraes et al, Biomaterials, 2009, 30, p. 5241 and Mehta et al, Anal. Chem., 2009, 81, p. 3714. However, the use of clear, flexible, non-UV-curable polyurethane to fabricate microfluidic devices, e.g., to decrease absorption of molecules, has not been demonstrated.

In accordance with the invention, a subclass of these polyurethane polymers, namely, ether-based aliphatic polyurethanes, has been identified for use in a microfluidic device, e.g., to decrease absorption of molecules. Ether-based aliphatic polyurethanes are aliphatic polymers consisting of isocynates and polyols joined by urethane links. In some embodiments, isocynates and/or polyols can be synthetic or naturally occurring.

In some embodiments, the isocynates can comprise at least one aliphatic isocynate. In one embodiment, the aliphatic isocynates can comprise dicyclohexylmethane-4,4′-diisocyanate, derivatives and/or isomers thereof.

In some embodiments, the polyols can be aromatic, semi-aromatic or aliphatic. In some embodiments, the polyols can comprise at least one aliphatic polyol. In various embodiments, the polyols can comprise polyethers (e.g., polyethylene glycol, poly(tetramethylene ether) glycols), polyesters (e.g., polyglycolic acid), derivatives or isomers thereof, or any combinations thereof.

In one embodiment, the ether-based aliphatic polyurethane comprises dicyclohexylmethane-4,4′-diisocyanate, or a derivative or an isomer thereof.

In some embodiments, the ether-based aliphatic polyurethane can be optically clear. The term “optically clear” is used herein to generally describe a material that is capable of being seen through based upon unaided, visual inspection. In accordance with the invention, this observation corresponds to a minimum transmission of light, that is, a light transmission of at least about 70%, at least about 75%, at least about 80%, at least about 90%, at least about 95%, at least about 96%, at least about 98% or higher. In one embodiment, the term “optically clear” refers to a 100% light transmission.

In some embodiments, the ether-based aliphatic polyurethane can be colorless or lack of color. As used herein, the term “colorless” refers to ether-based aliphatic polyurethanes lacking of sufficient color so as to be deemed transparent and clear either visually or by instrumentation. When visually evaluated, the term “colorless” does not mean that there is no color but, rather, the color is either not visually detectable or minimally detectable such that the viewer sees a clear material.

In some embodiments, the ether-based aliphatic polyurethane can be cured after mixing a curable composition. The phrase “a curable composition” is used in reference to a composition of ether-based aliphatic polyurethane that is polymerizable or cross-linkable through functional groups, e.g., by at least one method that includes, but is not limited to, temperatures, curing catalysts or curing accelerators, electron beam, chemical free-radical initiation, and/or photo-initiation such as by exposure to ultraviolet light or other actinic radiation.

The term “cured” or “curing” as used herein generally refers to at least a partial change in state, condition, and/or structure of a polymer. In some embodiments, the term “cured” or “curing” refers to gelling, toughening or hardening of a polymer, e.g., by cross-linking or polymerizing a polymer chains. The term “cured” with respect to a curable composition means that at least a portion of the polymerizable and/or crosslinkable components that form the curable composition is polymerized and/or crosslinked, e.g., at least about 50% curing, at least about 60% curing, at least about 70% curing, at least about 80% curing, at least about 90% curing, at least about 95% curing, at least about 98% curing or higher. In one embodiment, a curable composition is completely cured, when further curing results in no significant change in the polymer properties, such as hardness.

In some embodiments, the ether-based aliphatic polyurethane can be cured in the presence of curing catalysts and/or curing accelerators that are known in the art. In some embodiments, the ether-based aliphatic polyurethane can be cured in the absence of photo-initiation, e.g., UV light exposure.

The ether-based aliphatic polyurethane can be cured at any temperatures. In some embodiments, the ether-based aliphatic polyurethane can be cured at room temperature or higher, e.g., at least about 20° C., at least about 30° C., at least about 40° C., at least about 50° C., at least about 60° C., at least about 70° C., at least about 80° C., at least about 90° C. or higher. In some embodiments, the ether-based aliphatic polyurethane can be cured at room temperatures. In some embodiments, the ether-based aliphatic polyurethane can be cured at about 80° C. or higher.

The ether-based, aliphatic polyurethane can be cured for any period of time. In some embodiments, the ether-based aliphatic polyurethane can be cured for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours or longer. Depending on the curing conditions, e.g., curing temperatures, one of skill in the art can adjust the curing duration accordingly. For examples, the ether-based aliphatic polyurethane can be cured at room temperature overnight or at higher temperatures (e.g., about 80° C.) for a shorter period of time, e.g., about 2 hours.

In some embodiments, the ether-based, aliphatic polyurethane can be the bulk material of the microfluidic module described herein. In some embodiments, the ether-based, aliphatic polyurethane can coat at least one surface of one or more fluidic elements described herein.

### Decreasing Absorption of Molecules

In the microfluidic modules and methods described herein, the ether-based aliphatic polyurethane can decrease or inhibit absorption of molecules. A further aspect described herein is an ether-based aliphatic polyurethane for use in inhibiting absorption of molecules in a microfluidic device, wherein at least a portion of the ether-based, aliphatic polyurethane is in fluid communication. Such “molecules” refer to natural or synthetic molecules including, but are not limited to, drugs, biologics, steroids, contrast agents, fluorescent dyes, proteins, peptides, antibodies or fragments thereof, antibody-like molecules, and any combinations thereof.

The term “drugs,” as used herein, is art-recognized and refers to any chemical moiety that is a biologically, physiologically, or pharmacologically active substance that acts locally or systemically in a subject. Examples of drugs, also referred to as “therapeutic agents,” are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Examples of drugs include, but are not limited to, anti-AIDS substances, anti-cancer substances, antibiotics, immunosuppressants, anti-viral substances, enzyme inhibitors, including but not limited to protease and reverse transcriptase inhibitors, fusion inhibitors, neurotoxins, opioids, hypnotics, anti-histamines, lubricants, ranquilizers, anti-convulsants, muscle relaxants and anti Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents.

Without limitations, additional examples of drugs include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, proteins, antibodies or antibody-like molecules, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals. The term “drugs” also includes compounds that have the indicated properties that are under research and/or development, or not yet available in the U.S. The term “drug” includes pro-active, activated, and metabolized forms of drugs.

The term “biologics” as used herein refers to cells and/or biomolecules.

As used herein, the term “cells” refers to nucleated cells (i.e., cells containing one or more nuclei) or anucleated cells (i.e., platelets and red blood cells; cells that have no nucleus). Cells can be derived from any tissues or organs. In addition, cells can be modified, for example, cell lines, recombinant cells or hybridomas. In some embodiments, cells can include any eukaryotic cells, such as animal cells and/or plant cells. In some embodiments, cells can also encompass prokaryotic cells, such as bacteria and single-celled organisms.

As used herein, the term “biomolecules” refers to any protein, nucleic acids, siRNAs, microRNAs, carbohydrate, lipid, or any molecule, produced or existing free in body/biological fluids. Biomolecules can be present alone, or in combination with other biomolecules and/or cells, such as plasma products (i.e., blood cells, biomolecules, and salts). Biomolecules can also include, for example, antibodies and peptides, or compositions of biomolecules such as, for example, the proteins, peptides, and other biological organic molecules in plasma.

The term “nucleic acids” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants and analogs of either RNA or DNA made from nucleotide analogs, and, single (sense or antisense) and double-stranded polynucleotides.

The term “short interfering RNA” (siRNAs), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell. siRNA molecules can also be generated by cleavage of double stranded RNA, where one strand is identical to the message to be inactivated. The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense 60 strand and/or the antisense strand. The term “siRNA” includes duplexes of two separate strands, as well as single strands that can form hairpin structures comprising a duplex region.

The terms “microRNAs” and “miRNAs” as used interchangeably herein refer to any type of interfering RNA, including but not limited to, endogenous microRNA and artificial microRNA. Endogenous microRNAs are small RNAs naturally present in the genome which are capable of modulating the productive utilization of mRNA. Some of the endogenous microRNAs can regulate the expression of protein-coding genes at the post-transcriptional level. The term “artificial microRNA” includes any type of RNA sequence, other than endogenous microRNA, which is capable of modulating the productive utilization of mRNA. In some embodiments, the microRNAs are short ribonucleic acid (RNA) molecules, e.g., at least about 10 nucleotides long, at least about 15 nucleotides long, at least about 20 nucleotides long or longer. In some embodiments, the microRNAs are short RNA molecules, on average about 22 nucleotides long.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′)2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

The expression “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (Pl\ddot{ν}ckthun, The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, P0:6444-6448 (1993)).

“Contrast agents” are any chemical moieties that can be used to increase the degree of difference between the lightest and the darkest parts, e.g., during microscopy or imaging. For example, contrast agents or dyes include, without limitations, iodine, gadolinium or cyanine; enzymes such as horse radish peroxidase, GFP, alkaline phosphatase, or β-galactosidase; fluorescent dyes such as europium derivatives; luminescent substances such as N-methylacrydium derivatives; and any combinations thereof.

As used herein, the term “fluorescent dyes” refers to chemical moieties that, upon excitation by light energy of a particular wavelength or wavelengths, emit light at another wavelength or that emit light when paired with an appropriate excited donor fluorophore. Exemplary fluorescent dyes include, but are not limited to, any fluorescent dyes in the rhodamine, europium, fluorescein, coumarin, naphthalimide, benzoanthene, oxazone and acridine dye families, and derivatives thereof. In some embodiments, the fluorescent dyes can be lipophilic stains, e.g., Nile Red. Fluorescent dyes also include the ones that are commercially available, e.g., from Invitrogen or ThermoScientific. Without limitations, additional fluorescent dyes include FLUO-3, FURA-2, INDO-1 QUIN-2 and related compounds available from Molecular Probes; fluorescent pH indicators such as SNAFL, SNARF and related pH indicators; fluorescent cell viability indicators such as CALCEIN-AM and ethidium homodimer.

In certain embodiments of the microfluidic modules and methods described herein, the ether-based aliphatic polyurethane can decrease or inhibit absorption of hydrophobic molecules. The term “hydrophobic”, as used herein, refers to a characteristic of a molecule or part of a molecule which is non-polar and/or is immiscible with charged and polar molecules, and/or has a substantially higher dissolvability in nonpolar solvents as compared with their dissolvability in water and other polar solvents. The term “dissolvability” refers to either a complete or partial dissolution of molecules in a substance, e.g., a solvent. In some embodiments, the term “dissolvability” refers to maximal saturation concentration of molecules in a substance, e.g., a solvent, and the rest of the molecules remain as a suspension of small particles in the substance. Without wishing to be bound by theory, when in water, hydrophobic molecules can cluster together to form lumps, agglomerates, aggregates or layers on one of the water surfaces (such as bottom or top). Exemplary hydrophobic molecules include, without limitations, molecules comprising one or more alkyl groups, such as oils and fats, one or more aromatic groups, such as polyaromatic compounds, and/or one or more non-polar groups.

The term “absorption” generally refers to a process in which atoms, molecules or ions enter a bulk phase, for example, a gas, liquid or solid material. The term “absorption” as used herein refers to molecules dispersed in one material partitioning into another material. In one embodiment, the partitioning of molecules is based on the intermolecular interaction of molecules between two different materials. The intermolecular interaction of molecules with a material can be polar, non-polar, hydrophobic, hydrophilic, or any combinations thereof. In some embodiments, the intermolecular interaction of molecules with a material can be polar or hydrophilic. In some embodiments, the intermolecular interaction of molecules with a material can be non-polar or hydrophobic. In other embodiments, the partitioning of molecules is based on the relative solubility of the molecules between two different materials. The term “absorption” as used herein can encompass extracting, isolating or separating molecules from a material into another material. In some embodiments, the term “absorption” as used herein can encompass molecules depositing onto a surface. In accordance with the invention, a microfluidic module fabricated from at least one ether-based aliphatic polyurethane polymer decreases or inhibits partitioning of molecules from a fluid into the bulk polymer. In some embodiments, a microfluidic module fabricated from at least one ether-based aliphatic polyurethane polymer decreases or inhibits deposition of molecules from a fluid onto a surface of the polymer.

The terms “decrease,” “decreasing,”, “inhibit,” and “inhibiting” are all used herein generally to mean a decrease by a statistically significant amount. In some embodiments, the term “decrease” or “inhibit” as used herein refers to a decrease in absorption of molecules by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference level), or any decrease between 10-100% as compared to a reference level.

As used herein, the term “reference level” in reference to absorption of molecules means a degree of absorption of molecules occurred in a material other than ether-based aliphatic polyurethanes. Examples of such material include, but are not limited to, poly(dimethylsiloxane) (PDMS), silicon, glass, a silica-based substrate, quartz, polysilicon, gallium arsenide, polymethylmethacrylate (PMMA), polyvinylchloride (PVC), polystyrene polysulfone, polycarbonate, polymethylpentene, polypropylene, polyethylene, polyvinylidine fluoride, and ABS (acrylonitril-butadiene-styrene copolymer), polyurethane polymers that are not ether-based or aliphatic, and any combinations thereof.

### Exemplary Applications of Microfluidic Modules

Using various embodiments of the microfluidic modules described herein, a solution containing one or more contrast agents described herein can be flowed through at least one fluidic element. These contrast agents can be used to stain cells that exhibit certain surface proteins or that are producing particular biomolecules. In such embodiments, the substrate of the microfluidic modules can decrease absorption of at least one contrast agent and thus decrease background noise, e.g., autofluorescence due to partitioning of at least one contrast agent into the substrate. Accordingly, the microfluidic modules can provide higher detection sensitivity.

Some embodiments of the microfluidic modules described herein can be used to screen potential drugs or therapeutic agents described herein. In accordance with the invention, microfluidic modules described herein can decrease absorption of candidate drugs or therapeutic agents flowing through at least one fluidic element. Accordingly, the candidate drugs or therapeutic agents will be readily available to cells cultured in the microfluidic modules, e.g., to determine the physiological or therapeutic effect on the cells.

The microfluidic modules described herein can also be utilized in combination with at least one device or instrument, e.g., for viewing the effect of the candidate drugs on the cells. The instrument in one embodiment can comprise a microscope for viewing the effect.

Exemplary embodiments of the microfluidic module and method of making the same can be also described by any one of the following numbered paragraphs.
1. A microfluidic module comprising a substrate and at least one fluidic element disposed therein, wherein the substrate comprises at least one ether-based, aliphatic polyurethane; and wherein at least a portion of the at least one ether-based, aliphatic polyurethane is in fluid communication.
2. The microfluidic module of paragraph 1, wherein the at least one ether-based, aliphatic polyurethane decreases absorption of molecules.
3. The microfluidic module of paragraph 2, wherein the molecules are selected from the group consisting of drugs, biologics, contrast agents, fluorescent dyes, proteins, peptides, antibodies, and any combinations thereof
4. The microfluidic module of paragraph 2 or 3, wherein the molecules are hydrophobic molecules.
5. The microfluidic module of any of paragraphs 1-4, wherein the at least one ether-based, aliphatic polyurethane is optically clear.
6. The microfluidic module of any of paragraphs 1-5, wherein the at least one ether-based, aliphatic polyurethane is colorless.
7. The microfluidic module of any of paragraphs 1-6, wherein the at least one ether-based, aliphatic polyurethane is cured after mixing a curable composition.
8. The microfluidic module of any of paragraphs 1-7, wherein the at least one ether-based, aliphatic polyurethane is adapted for replica molding.
9. The microfluidic module of any of paragraphs 1-8, wherein the at least one ether-based, aliphatic polyurethane comprises dicyclohexylmethane-4,4′-diisocyanate, a derivative or an isomer thereof.
10. The microfluidic module of any of paragraphs 1-9, wherein the at least one fluidic element is a microwell.
11. The microfluidic module of any of paragraphs 1-9, wherein the at least one fluidic element is a microchannel.
12. The microfluidic module of any of paragraphs 1-11, further comprising at least one cell.
13. A method of making a microfluidic module, comprising forming a microfluidic module from at least one ether-based, aliphatic polyurethane, wherein the microfluidic module comprises a substrate and at least one fluidic element disposed therein; and wherein at least a portion of the at least one ether-based, aliphatic polyurethane is in fluid communication.
14. The method of paragraph 13, wherein the microfluidic module is formed by replica molding.
15. The method of paragraph 13, wherein the microfluidic module is formed by micromachining
16. The method of paragraph 13, wherein the microfluidic module is formed by solid-object printing.
17. The method of any of paragraphs 13-16, wherein the at least one ether-based, aliphatic polyurethane decreases absorption of molecules.
18. The method of paragraph 17, wherein the molecules are selected from the group consisting of drugs, contrast agents, fluorescent dyes, proteins, peptides, antibodies, and any combinations thereof
19. The method of any of paragraphs 17-18, wherein the molecules are hydrophobic molecules.
20. The method of any of paragraphs 13-19, wherein the at least one ether-based, aliphatic polyurethane is optically clear.
21. The method of any of paragraphs 13-20, wherein the at least one ether-based, aliphatic polyurethane is colorless.
22. The method of any of paragraphs 13-21, wherein the at least one ether-based, aliphatic polyurethane is cured after mixing a curable composition.
23. The method of any of paragraphs 13-22, wherein the at least one ether-based, aliphatic polyurethane comprises dicyclohexylmethane-4,4′-diisocyanate, a derivative or an isomer thereof.
24. The method of any of paragraphs 13-23, further comprising coating the at least one fluidic element with cell adhesion molecules.
25. The method of any of paragraphs 13-24, wherein the at least one fluidic element is a microwell.
26. The method of any of paragraphs 13-24, wherein the at least one fluidic element is a microchannel.
27. An ether-based, aliphatic polyurethane for use in inhibiting absorption of molecules in a microfluidic module, wherein at least a portion of the ether-based, aliphatic polyurethane is in fluid communication.
28. The ether-based, aliphatic polyurethane of paragraph 27, wherein the molecules are hydrophobic molecules.

### Some Selected Definitions

Unless defined otherwise, all technical and scientific terms used herein have the same meaning as those commonly understood to one of ordinary skill in the art to which this invention pertains. Although any known methods, devices, and materials can be used in the practice or testing of the invention, the methods, devices, and materials in this regard are described herein.

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein the term “comprising” or “comprises” is used in reference to compositions, methods, and respective component(s) thereof, that are useful in an embodiment described herein, yet open to the inclusion of unspecified elements, whether useful or not for the embodiment.

As used herein and in the claims, the singular forms “a”, “an” and “the” include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “derivative” as used herein refers to a chemical substance related structurally to another, i.e., an “original” substance, which can be referred to as a “parent” compound. A “derivative” can be made from the structurally-related parent compound in one or more steps. In some embodiments, the general physical and chemical properties of a derivative can be similar to or different from the parent compound.

As used here in the term “isomer” refers to compounds having the same molecular formula but differing in structure. Isomers which differ only in configuration and/or conformation are referred to as “stereoisomers.” The term “isomer” is also used to refer to an enantiomer.

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane polarized light in different directions). Enantiomers generally have identical physical properties, such as melting points and boiling points, and also have identical spectroscopic properties. Enantiomers can differ from each other with respect to their interaction with plane polarized light and with respect to biological activity.

The designations “R” and “S” are used to denote the absolute configuration of the molecule about its chiral center(s). The designations may appear as a prefix or as a suffix; they may or may not be separated from the isomer by a hyphen; they may or may not be hyphenated; and they may or may not be surrounded by parentheses.

The designations or prefixes “(+) and (−)” are employed to designate the sign of rotation of plane-polarized light by the compound, with (−) meaning that the compound is levorotatory (rotates to the left). A compound prefixed with (+) is dextrorotatory (rotates to the right).

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

The examples presented herein relate to the use of ether-based aliphatic polyurethanes to decrease absorption of molecules, e.g., hydrophobic molecules, in a microfluidic module.

### Example 1

### Inhibition of Hydrophobic-Molecule Absorption on Ether-Based Aliphatic Polyurethanes

Strong absorption of small hydrophobic molecules such as drugs, fluorescent dyes, or cell signaling molecules in PDMS microfluidic devices can result in reduction of effective drug concentration, time-dependent changes in compound concentrations, cross-contamination, lower detection sensitivity, and higher background fluorescence. Partitioning of molecules into the bulk is in part behind the slow industrial acceptance of PDMS microfluidic devices. While there are clear and flexible materials such as perfluoropolyethers that make inroads into the fabrication of microfluidic devices, they still suffer from absorption of small hydrophobic molecules (Devaraju and Unger, Lab Chip, 2011 11, p. 1962).

Polyurethanes are a very broad class of polymers comprised of the isocynate and the polyol groups. They have been used with success in many industries including the medical industry. A subclass of these polymers that do not significantly absorb small hydrophobic molecules, but that are optically clear, flexible, and that can be processed by replica molding in a basic laboratory setting would be particularly appealing for both rapid prototyping and manufacturing of microfluidic devices for cell-based drug and toxin testing applications. Recently, thin polyurethane films have been integrated into PDMS or rigid polymer devices. See for example, C. Moraes et al., Biomaterials, 2009, 30, p. 5241 and G. Mehta et al., Anal. Chem., 2009, 81, p. 3714. However, clear and flexible polyurethane microfluidic devices fabricated by replica molding have not been demonstrated. Here the inventors describe performance of a castable polyurethane that is similar to PDMS in terms of optical transparency and flexibility, but is drastically different regarding absorption of small hydrophobic molecules. The material allows for cell culture and device microfabrication by replication molding and corona or plasma bonding.

Polyurethane elastomer GSP 1552-2 was obtained from GS Polymers, Inc. The GSP 1552-2 elastomer is a two-part system that has a 15-minute gel time and cures overnight at room temperature or in about two hours at 80° C. The GSP 1552-2 elastomer is optically clear, flexible (determined to be shore 60 A in a hardness test) and can be used to fabricate a microfluidic device in a similar away to PDMS. However, the GSP 1552-2 elastomer is more hydrophilic than PDMS.

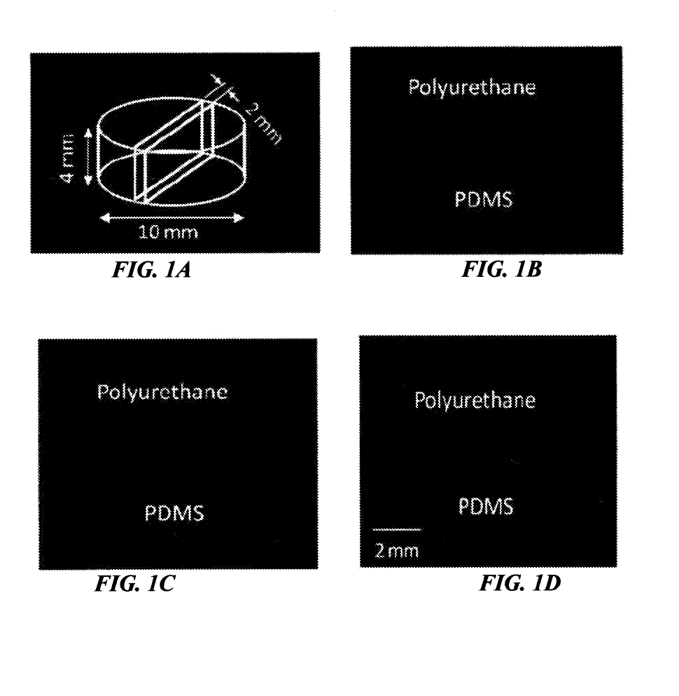

Side-by-side dye absorption studies of poly(dimethylsiloxane) (PDMS) and the ether-based aliphatic polyurethane were performed. To show fundamental differences in small molecule absorption, 10-mm diameter discs with a 4-mm thickness were punched from fully cured ether-based aliphatic polyurethane and PDMS blanks, and immersed in 1 mM solutions of Nile red, rhodamine B, and FITC solutions for 48 hours. The discs were then spray-rinsed with DI water, air-dried, and imaged from the top. Finally, a ˜2 mm-thick slice was sectioned from each disc and imaged from a cut side (FIG. 1A). The hydrophobic dye, Nile red, absorbed virtually into the entire bulk of the PDMS disc while the bulk of the ether-based aliphatic polyurethane disc was not affected (FIG. 1B). Rhodamine B partitions significantly into the bulk of PDMS but not into ether-based aliphatic polyurethane (FIG. 1B). FITC did not partition into either PDMS or ether-based aliphatic polyurethane elastomer (FIG. 1B).

### Example 2

### Demonstration of Ether-Based Aliphatic Polyurethane in Use for Molding and Fabricating a Microfluidic Device

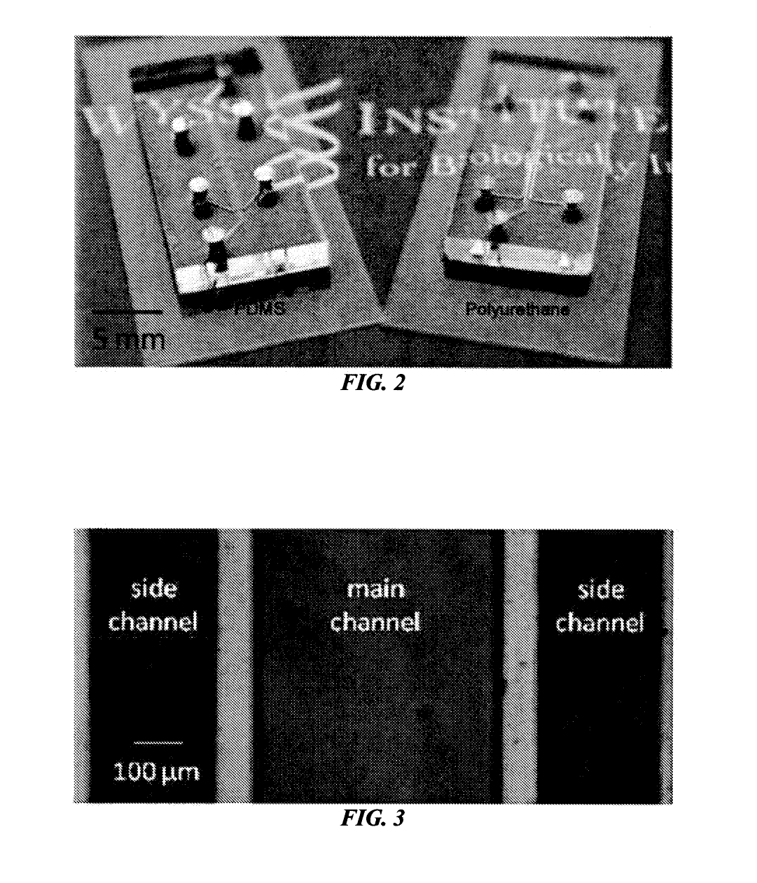

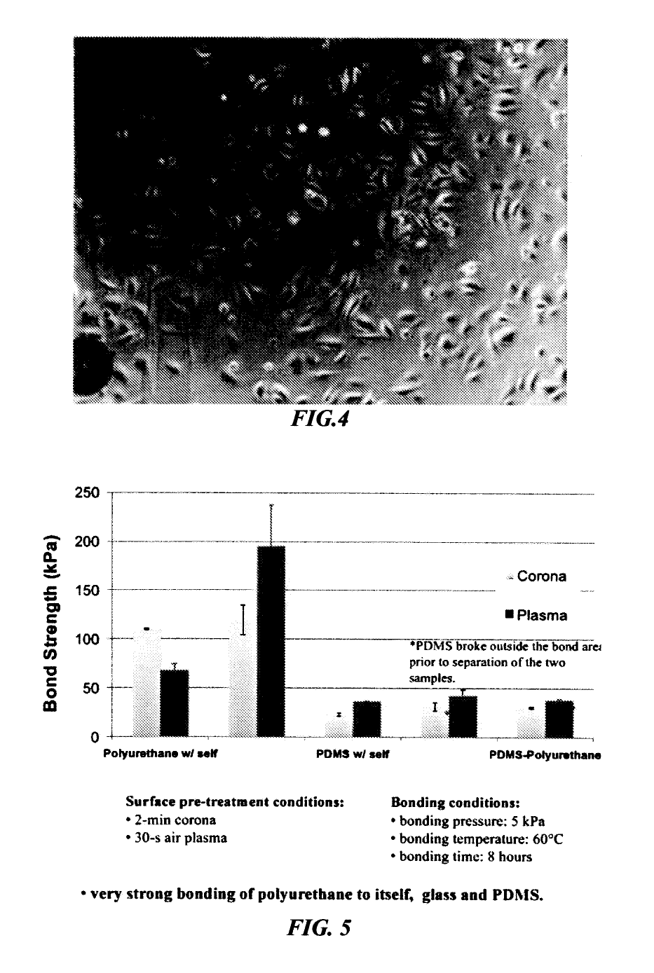

FIG. 2 shows side-by-side optically clear and flexible (60 Shore A) ether-based aliphatic polyurethane and PDMS microfluidic devices. Each device consists of a patterned layer corona-bonded to a glass cover slip. The PDMS device was cast directly from a silanized silicon wafer with SU-8 resist features. Because of a stronger adhesion of polyurethane to silanized silicon masters, the ether-based aliphatic polyurethane device was cast from a silicone mold replicated from the original silicon master. Photograph of microchannels of the corona-bonded ether-based aliphatic polyurethane device filled with food-colored aqueous solutions illustrating the feasibility of molding and bonding is shown in FIG. 3.

### Example 3

### In Vitro Culture on an Ether-Based Aliphatic Polyurethane Material

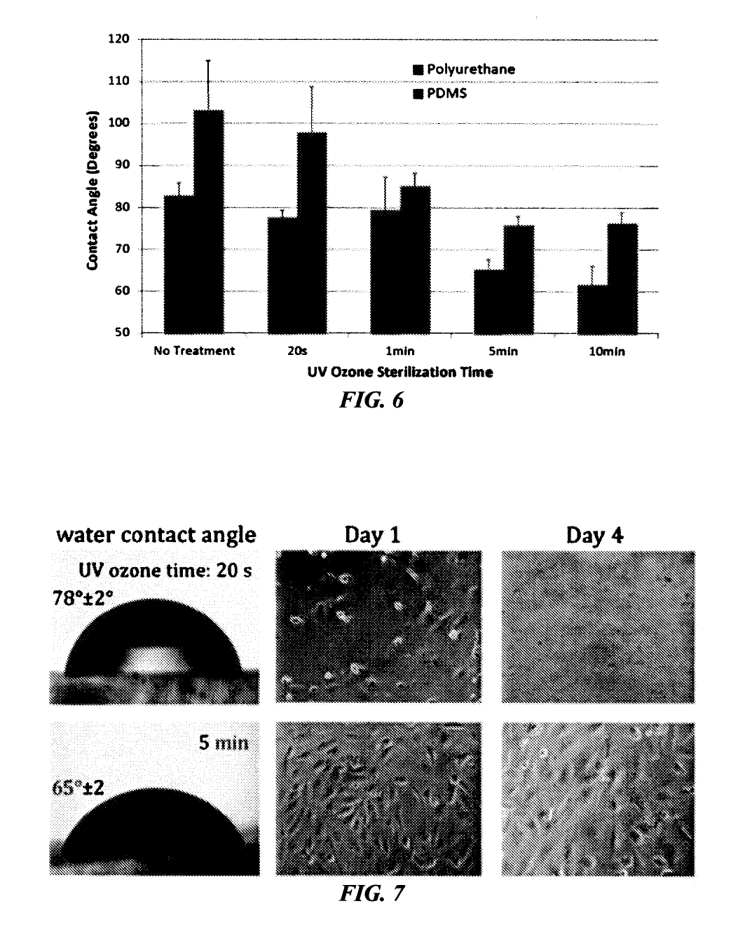

To demonstrate suitability of the polyurethane material for fabricating microfluidic cell culture devices, human umbilical vein endothelial cells were cultured on the fibronectin-coated ether-based aliphatic polyurethane discs and imaged (FIG. 4). Cell spreading and attachment for HUVEC cells was observed.

### Example 4

### Materials and Methods

GS Elastomeric Polyurethane:

The elastomeric polyurethane used was a castable two-component polymer GSP 1552-2 (GS Polymers, Inc.). The component 1552-2A is composed of dicyclohexylmethane-4,4′-diisocynate (up to 85% by weight) and prepolymer of dicyclohexylmethane-4,4′-diisocynate (15-20%). The component 1552-2B is a proprietary polyol blend (up to 99.9%) and the catalyst dibutyltin dilaurate (<0.5%).

For bonding and optical characterization, the components were mixed in a 1:1 weight ratio using a Planetary Centrifugal Mixer “Thinky Mixer” (ARE-310, Thinky) After mixing, an appropriate amount of polyurethane was poured onto mirror-polished aluminum surface with vertical barriers to generate a layer approximately 2 mm in thickness. Because centrifugal deaeration of the polyurethane at atmospheric pressure in the Thinky mixer was not satisfactory, the polymer was further degassed in desiccator at 698.5 mm of Hg for 30 minutes immediately after pouring the polymer into the aluminum mold. Curing was performed overnight at room temperature atmospheric pressure followed by curing at 60° C. for 2 hours.

For casting microfluidic polyurethane devices, GS polyurethane was mixed in Planetary Centrifugal Vacuum Mixer “Thinky Mixer” ARV-310LED that eliminated the degassing step. The 310 polyurethane molds were degassed in a desiccator prior to pouring the GS polyurethane into them.

310 polyurethane:

Castable 310 polyurethane (Smooth cast 310, Smooth-On Inc.) was mixed in 1:1 ratio in Planetary Centrifugal Vacuum Mixer “Thinky Mixer” ARV-310LED and cast into PDMS molds that were degassed in a desiccator.

PDMS:

The polydimethylsiloxane (PDMS) used was Sylgard 184 (Dow Corning). The silicone elastomer base and silicone elastomer curing agent were mixed in a 10:1 weight ratio in the ARE-310 “Thinky Mixer.” The PDMS was then cast, further degassed, and cured in the same manner as polyurethane.

Sample Preparation for Bond Strength Tests and Water Contact Angle Measurements:

After peeling off the polyurethane and PDMS sheets from the aluminum molds, an oblong 50.8 mm×6.4 mm punch was used to cut samples for the bond strength test and a circular 10 mm punch was used to create disks for the dye absorption test. Rectangular samples were cut for contact angle measurements. The surfaces that were in contact with the mirror-polished aluminum surface during casting were selected as active surface for bonding and water contact angle measurements.

Cleaning:

Before testing, all polyurethane and PDMS samples as well as glass slides used in the bonding tests were cleaned with a detergent solution (Natural Dish Liquid, Seventh Generation, Inc.) in a standard ultrasonic cleaner. The active ingredients of the detergent were sodium lauryl sulfate, caprylyl/myristyl glucoside, and lauramine oxide. They samples were then properly rinsed in MilliQ water and blow-dried. Glass slides were further rinsed with acetone.

Corona Treatment:

Both PDMS and polyurethane were treated with a high-frequency corona generator (model BD-20AC, Electro-Technic Products, Inc.). The corona generator tip was scanned 5 mm above both surfaces to be bonded.

Plasma Treatment:

Plasma treatment of samples was performed with an air plasma cleaner (SPI Plasma-Prep II Plasma Etcher, SPI Supplies, Inc.). The samples on a glass slide were placed into the barrel with the bonding surfaces facing up and the treatment was done at the pressure of 380 mTorr of air and power of 10 W.

UV/Ozone Treatment:

UVO treatment of samples was done with a UVO-cleaner (model 342, Jelight Co., Inc.) equipped with a low pressure mercury vapor grid. The samples were placed with test surfaces facing up at the distance of 5 mm from the UV light lamp, as recommended by the manufacturer. At this distance, the stated intensity at 184.9 and 253.7 nm is approximately 6 mW/cm² and 30 mW/cm², respectively.

Bond Strength Testing:

Immediately after surface treatment, the 50.8 mm×6.4 mm oblong samples were overlapped approximately 25×6.4 mm and pressed together with an approximate pressure of 1 kPa. Then the samples were placed in 40° C. or 60° ovens with the appropriate weights placed on top of the overlapping bonding region to achieve bonding pressures of 3.5 kPa, 7 kPa, 14 kPa. After removal from the oven, the bonded samples were allowed to cool before bond strength testing.

Bond strength testing was performed with a tensometer (Model 5544, Instron). The free ends of the partially overlapped and bonded oblong samples were clamped by the two pneumatic grips of the tensometer. The samples were stretched at the separation rate of 0.2 mm/second, until the bond failed. The maximum load supported by the bond was recorded and normalized by bond area to give the bond strength of each sample in Pascals (N/m²). If a sample failed at a location other than the bond area then the minimum value for the bond strength was given.

Water Contact Angle Testing:

The water contact angle was measured using the static sessile drop method. The in-house built measuring setup consisted of top plate (model 290-TP, Newport, Inc.) with a mounted diffuser (model DG10-1500, Thorlabs), an XYZ stage (461-series, Newport, Inc.), and a mirror (PF10-03-P01, Thorlabs) attached with a 45-degree optic holder (model H₄₅B2, Thorlabs) to a goniometer (model GN1, Thorlabs). Tilt of the mirror was adjusted with the goniometer to provide an appropriate viewing angle (0-3°). Small rectangular polymer samples were placed on a horizontal platform of the XYZ stage. A 1.5-μL water droplet was dispensed with a pipette on the polymer surface, illuminated with a gooseneck bright light source through a diffuser, and imaged immediately using a stereo microscope (Discovery V8, Carl Zeiss, Inc.). A digital image of the droplet was analyzed with ImageJ software and the DropSnake module (A. F. Stalder, G. Kulik, D. Sage, L. Barbieri, P. Hoffmann).

### Results and Discussion

Optimization of Corona Pre-treatment Time and Bonding Conditions for Bonding Polyurethane to Polyurethane:

Optimization of polyurethane surface pre-treatment and polyurethane to polyurethane bonding conditions was done in two stages. In the first stage, the inventors determined the working bounds by testing several coarsely spaced corona pre-treatment and annealing times and qualitatively evaluating the bond strength and visual changes in the sample appearance. In the second stage, inventors maintained the selected corona pre-treatment time constant and quantified the shear bond strength as a function of bonding pressure, annealing temperature, and a narrowed range of annealing time.

First, the inventors varied corona pre-treatment time and annealing time, while keeping the bonding pressure and annealing temperature constant at 14 kPa and 60° C., respectively. The inventors discovered that both longer corona treatment times (˜5 min) and annealing times (above 24 hours) lead to a strong bond but also a pronounced yellowing of the polyurethane. In contrast, shorter corona pre-treatment times (˜1 min) combined even with long annealing times (above 24 hours) resulted in a weak bond. Optimal results were obtained for two-minute corona pre-treatment time that provided a strong bond without the yellowing effects. The next step was to determine minimum bonding pressure, lowest annealing temperature and shortest annealing time. Establishing these conditions is desirable to avoid potential distortion of imprinted features and material degradation, and to keep the overall bonding time at minimum. After performing a set of preliminary tests the inventors investigated bonding pressures of 3.5 kPa, 7 kPa, 14 kPa, annealing temperatures of 23.7° C., 40° C., 60° C., and annealing times of 2 hours, 4 hours, 8 hours. Following pre-treatment and bonding, all 27 possible permutations of the three parameters were evaluated by measuring shear bond strength with Instron 5544 tensometer. The measurements revealed that varying the bonding pressure within the investigated range does not have a significant effect on the bond strength. The samples annealed at 60° C. had the strongest bond of the three temperature permutations, followed by samples annealed at 40° C. Room temperature bonding yielded the weakest bonds (data not shown). Longer annealing times resulted in a stronger bond. However, there seems to be some saturation of bond strength as the 8-hour sample was only 3% stronger than the 4-hour sample, while the 4-hour sample was 7% stronger than the 2-hour sample (data not shown).

Bond Strength between Corona and Plasma Pre-Treated Polyurethane, PDMS, and Glass Surfaces:

Because forming a strong bond between polyurethane and itself is crucial for fabricating multilayer polyurethane microfluidic devices and only a single material is involved, its bonding conditions were optimized first. This was done employing a commonly used low cost corona surface treatment technique. Next, to be able to fabricate hybrid polymer microfluidic devices with glass optical windows, the inventors applied the developed process to bonding polyurethane to polyurethane to bonding polyurethane to glass and polyurethane to PDMS. To provide a comparison framework, the inventors also measured shear bond strength between PDMS and itself and between PDMS and glass. However, we applied the bonding conditions optimized for polyurethane to PMDS, and thus, the conditions may not be optimal for PDMS. Finally, because plasma surface pre-treatment is also commonly used for bonding PDMS and other polymers, the inventors further investigated the bond strength of air plasma pre-treated polyurethane bonded to itself, glass, and PDMS using the conditions optimized for the corona pre-treated samples. The results are shown, together with water contact angle data, in Table 1 and FIG. 5.

TABLE 1
Bond strength and water contact angle of
1552-2 GS polyurethane and PDMS.

| Material | Surface | | | Bond Strength |
|---|---|---|---|---|
| Combination | Treatment* | Untreated | Treated | (kPa)** |
| Polyurethane- | Corona | 82.9 ± 3.0 | 60.9 ± 2.5 | 110.0 ± 1.0 |
| Polyurethane | | | | 119.4 ± 15.0 |
| Polyurethane- | | | | |
| Glass | | | | |
| Polyurethane- | Air Plasma | | 59.8 ± 2.3 | 68.4 ± 6.9 |
| Polyurethane | | | | 195.3 ± 42.2 |
| Polyurethane- | | | | |
| Glass | | | | |
| PDMS-PDMS | Corona | 103.1 ± 12.0 | 44.9 ± 3.3 | 23.2 ± 1.8 |
| PDMS-Glass | | | | >31.2 ± 4.1*** |
| PDMS-PDMS | Air Plasma | | 4.1 ± 1.0 | >36.6 ± 0.3*** |
| PDMS-Glass | | | | >42.9 ± 6.0*** |
| Polyurethane- | Corona | see above | see above | 30.3 ± 1.0 |
| PDMS | Air Plasma | | | >38.1 ± 1.2*** |

*The surfaces were pre-treated either with corona for 2 minutes or with air plasma for 30 seconds.
**The samples were bonded at the bonding pressure of 5 kPa, bonding temperature of 60° C., and bonding time of 8 hours.
***PDMS broke outside the bond area prior to separation of the two samples.

As the data in Table 1 shows, a strong bond can be achieved between polyurethane and itself, polyurethane and glass, and polyurethane and PDMS. The versatile bonding provides flexibility for the fabrication of hybrid polyurethane microfluidic devices. It can be also seen from Table 1 that corona pre-treatment outperforms air plasma pre-treatment for bonding polyurethane to itself but underperforms air plasma treatment for bonding polyurethane to glass, even though water contact angles of the polyurethane surfaces were in both cases approximately the same (60-61°). Further, for corona pre-treated samples, bond strength of corona pre-treated polyurethane bonded to itself was stronger both than the bond strength of polyurethane to PDMS and PDMS bonded to itself.

The Effect of UV/Ozone Sterilization on Static Water Contact Angle of Polyurethane and PDMS:

As a step for preparing polymer materials as scaffolds for cell culture, they are usually sterilized and coated with an extracellular matrix protein. Because sterilization of polyurethane by autoclaving is not feasible due to an elevated temperature of the process (typically 121°-134°, the inventors used a low-temperature UV ozone sterilization technique (UVO). In addition to sterilizing the polymer, the UVO treatment has several other important effects on the polymer. One of them is a change in water contact angle (FIG. 6). This is often used for improving cell attachment to for example PDMS and can be exploited for improving cell culture conditions on polyurethane as well.

The Effect of UV/Ozone Treatment of Cell Adhesion:

The effect of UVO treatment of polyurethane on the adhesion of human umbilical endothelial cells (HUVECs) was determined. is The 1552-2 GS Polyurethane (1552-2 GS) was sterilized by UV ozone and 9.5×3 mm samples inserted into a 48 well-plate. Samples were treated in 20 μg/ml fibronectin solution in 50 mM carbonate buffer at pH 9.3 and 4° C. for 24 h and HUVECs seeded at 2×105 cells/well. As shown in FIG. 7, extending the time of UVO treatment from 20 s (time recommended by the manufacturer for sterilization of samples) to 5 minutes had a dramatic effect on cell attachment. Longer sterilization lead to an increase in cell attachment.

Cell Cultures:

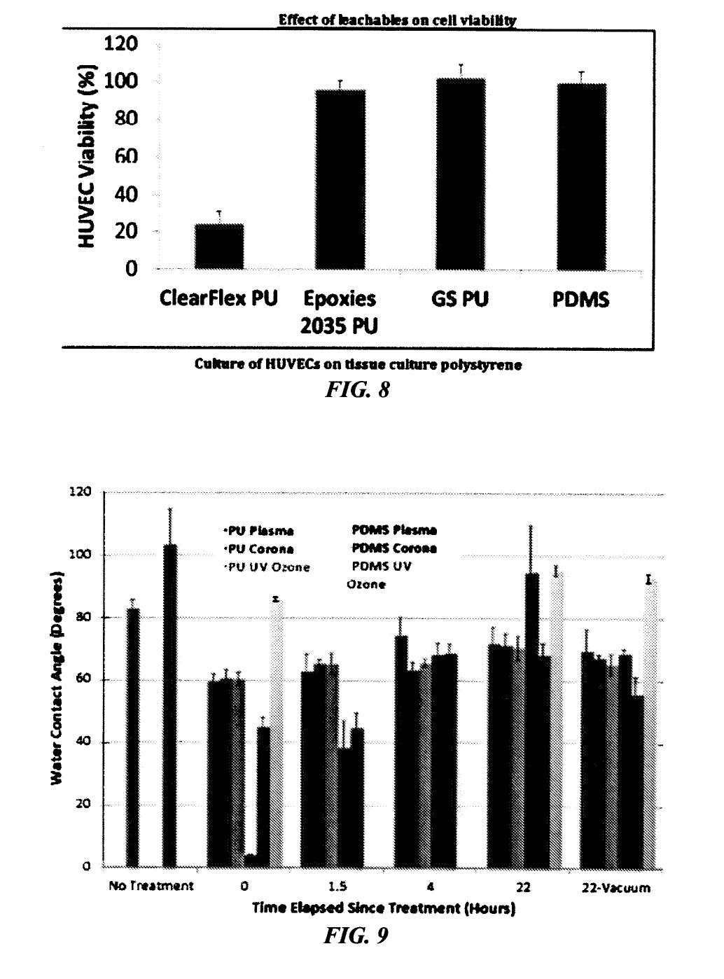

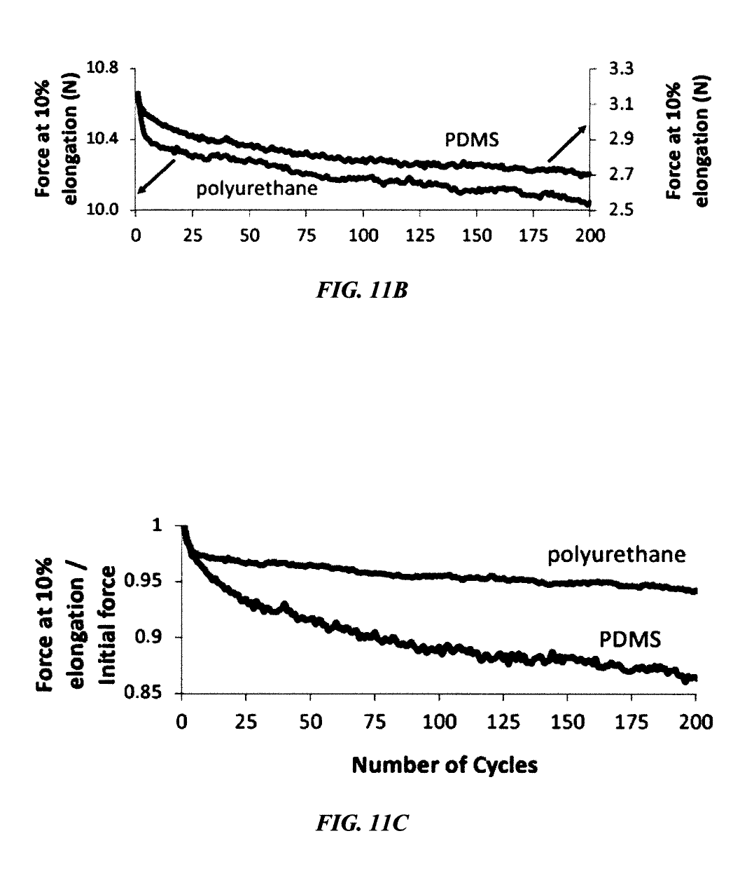

Effect of different materials on cells was also determined. Shredded polymer, 1 g, was incubated in 3 mL of water at 37° C. for several days. Small amounts of leachant solutions were added to human umbilical vein endothelial cells cultured to confluency in 96-well tissue culture plate. Cells were exposed to leachants for 24 hours and cell viability evaluated with MTT assay. HUVEC viability was high with epoxies 2035 PU, GS PU and PDMS as compared to ClearPlex PU (FIG. 8) FIG. 11.

Hydrophobic Recovery of Polyurethane Treated with Air Plasma, Corona Discharge, and UV Ozone:

Hydrophobic recovery of polyurethane treated with air plasma, corona discharge, and UV ozone are shown in comparison with PDMS in FIG. 9.

Optical Properties of Polyurethane:

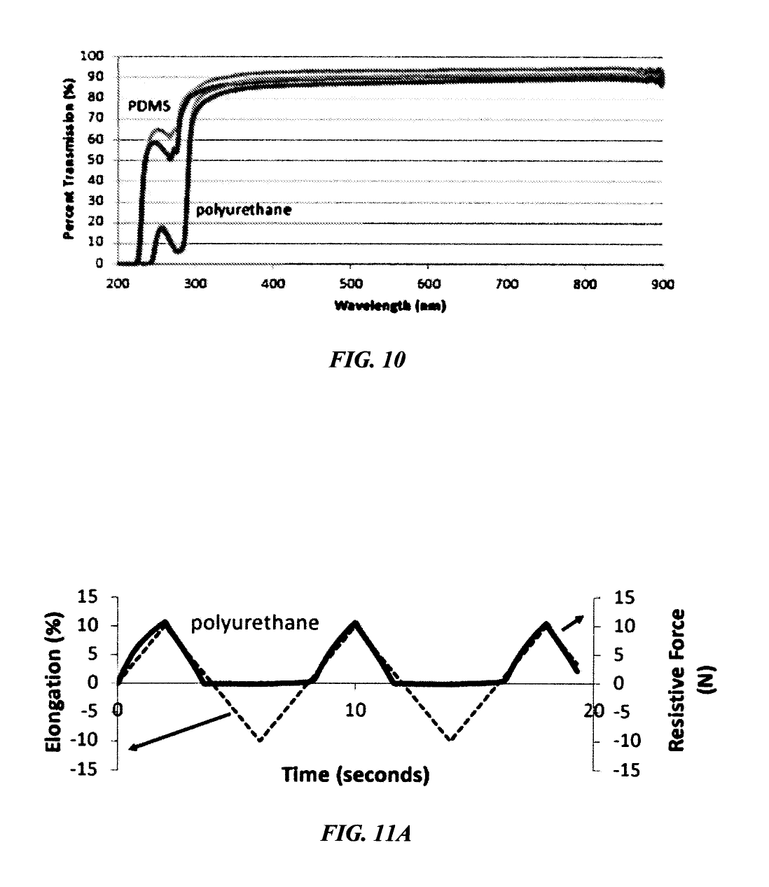

Optical transmission of polyurethane in the spectrum spanning from 200 to 900 nm is shown in FIG. 10. It can be seen that for the wavelengths above 300 nm the transmission of polyurethane is virtually identical to that of PDMS. However, below 300 nm, PDMS is more transparent than polyurethane.

Performance of Polyurethane Subjected to Cyclical Load Testing:

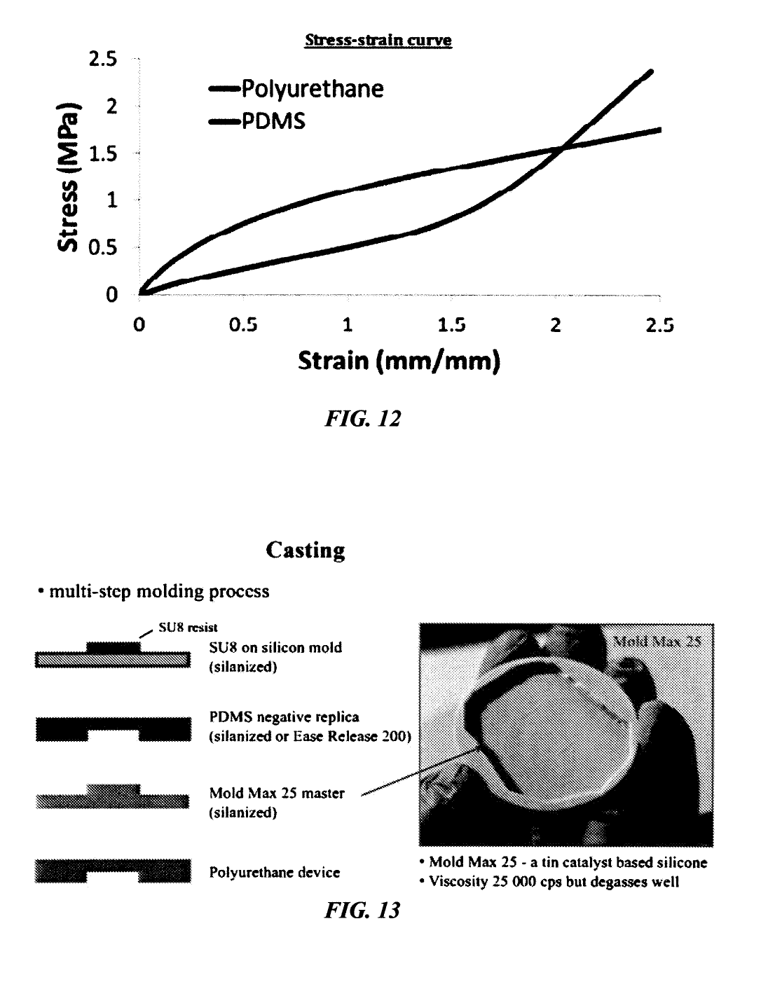

To investigate how well the polyurethane can recover after repeated stretching, the inventors elongated a sample by 10% with 8-second cycle period and measured the resistive force with a tensometer (FIG. 11A). Resistive force at 10% elongation as a function of number of cycles is shown in FIG. 11B. Resistive force normalized by the initial force is plotted in FIG. 11C. It can be seen from the normalized plot that polyurethane recovers from cyclic load very well and outperforms in this test PDMS. The stress strain curve of polyurethane and PDMS is shown in FIG. 12.

Casting of Elastomeric Polyurethane Microfluidic Devices:

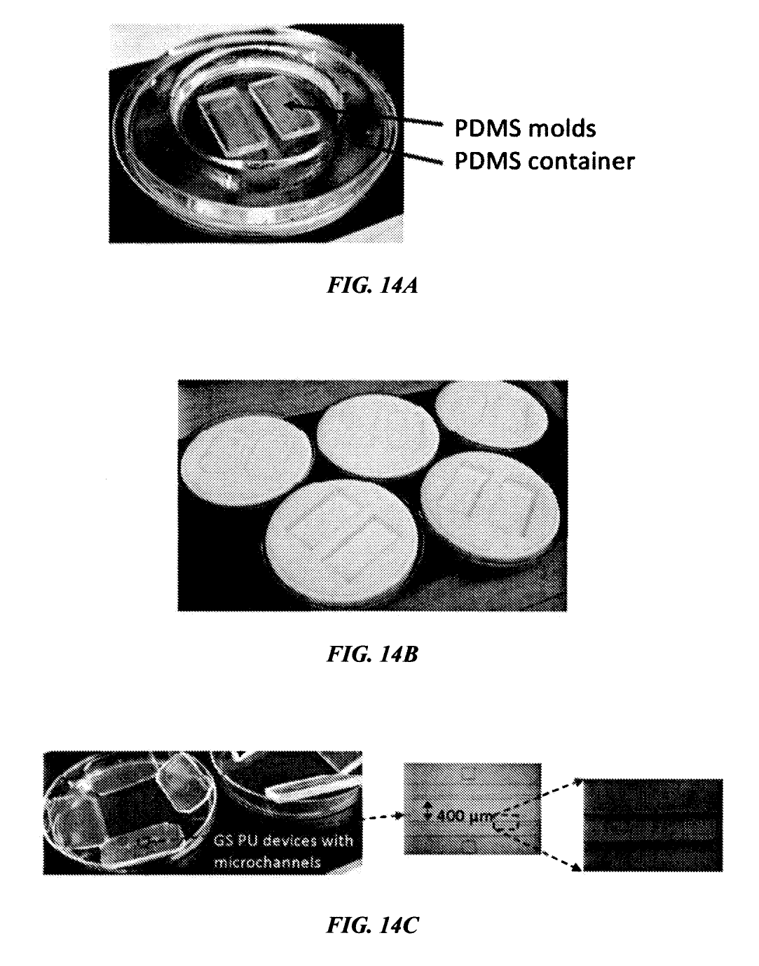

Because GS polyurethane adheres relatively strongly to silanized SU-8 on silicon masters, a multistep casting process can be used for device fabrication as shown in FIGS. 13 and 14. In one experiment, tin-based silicone was used to avoid inhibition of the polyurethane curing process (FIG. 13).

In another experiment, PDMS was first cast on SU-8 masters, cured, and peeled off (FIG. 14A). The PDMS replicas were then placed into a PDMS container and 310 polyurethane was cast on the PMDS, cured, and peeled off (FIG. 14B). This is a modification of a process developed for PDMS casting from rigid plastic masters as described in S. P. Desai et al, “Plastic masters-rigid templates for soft lithography,” Lab Chip, 2009, 1631-1637. The hard 310 polyurethane molds were silanized and elastomeric GS polyurethane was cast into the 310 polyurethane molds, cured, and peeled off (FIG. 14C).

Fabrication of Free Standing Porous Polyurethane Membranes:

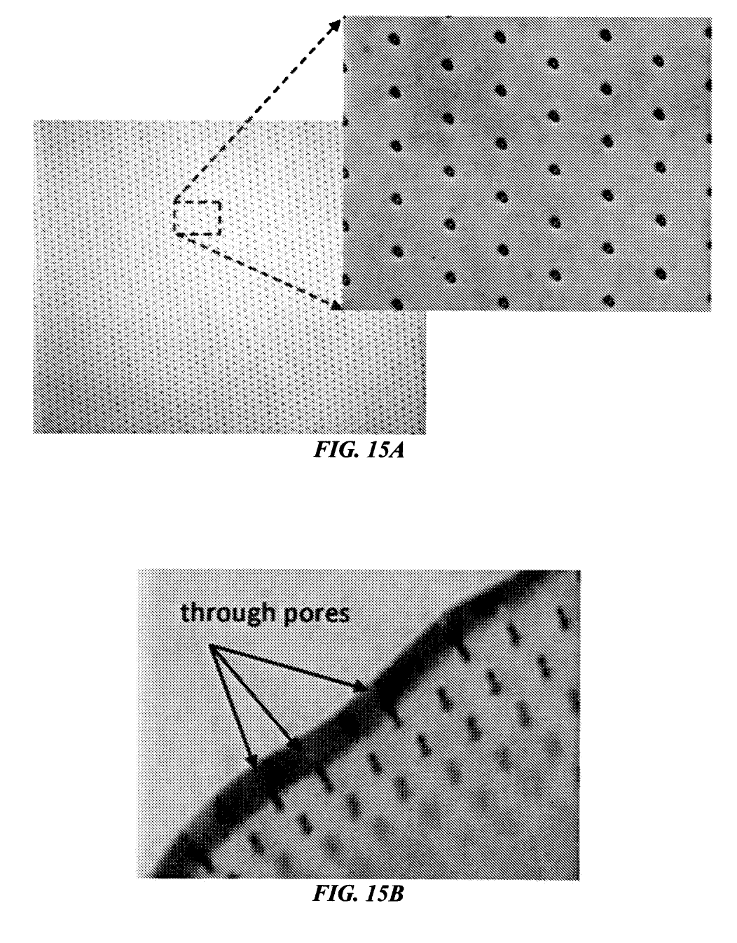

PDMS was cast on silanized silicon masters containing an array of pillars fabricated by deep reactive ion etching, cured, and peeled off. The PDMS mold is the negative of the silicon master, i.e., it contains an array of wells. The 310 polyurethane is cast into the PDMS mold with an array of wells, cured, and peeled off. This process results in a hard plastic replica of the original silicon master (see the process description above). Next, PMDS “handle” slabs are fabricated and plasma treated for 35 minutes. GS polyurethane is spin-coated on the PDMS slabs and the silanized 310 hard polyurethane masters with pillars are pressed on the spin-coated GS polyurethane. After curing, the hard 310 polyurethane masters are removed and the polyurethane membranes are peeled off or transferred to other parts. FIG. 15A shows patterned elastomeric GS polyurethane on PMDS “handle” slabs. The pores are approximately 7 μm in diameter. FIG. 15B shows a piece of freestanding GS polyurethane membrane, approximately 50 μm in thickness, after it has been peeled off from the PDMS slab.

Inventors have discovered a castable polyurethane that is similar to PDMS in terms of optical transparency and flexibility but drastically different regarding absorption of small hydrophobic molecules. They have shown that the material allows for cell culture and device microfabrication by replication molding and corona or plasma bonding. Polyurethane organs-on-a-chip microdevices find broad applicability in assays that involve cells and/or small hydrophobic molecules, and thus are valuable for drug discovery applications, toxin testing, fluorescence microscopy and cell signaling studies.

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

Further, to the extent not already indicated, it will be understood by those of ordinary skill in the art that any one of the various embodiments herein described and illustrated can be further modified to incorporate features shown in any of the other embodiments disclosed herein.

1. A microfluidic module comprising a substrate and at least one fluidic element disposed therein, wherein the substrate comprises at least ono first ether-based, aliphatic polyurethane; and wherein at least a portion of the at least one first ether-based, aliphatic polyurethane is in fluid communication.

2-28. (canceled)

29. The microfluidic module of claim 1, wherein the substrate comprises a first layer and a second layer, the first layer comprising the first ether-based, aliphatic polyurethane, and the second layer comprising second ether-based, aliphatic polyurethane, glass, polydimethylsiloxane (PDMS), or any combinations thereof.

30. The microfluidic module of claim 29, wherein when the substrate comprises PDMS, the PDMS is excluded from fluid communication.

31. The microfluidic module of claim 1, wherein the first ether-based, aliphatic polyurethane is characterized by a decreased absorption of molecules thereon.

32. The microfluidic module of claim 31, wherein the molecules are selected from the group consisting of drugs, biologics, contrast agents, fluorescent dyes, proteins, peptides, antibodies, and any combinations thereof.

33. The microfluidic module of claim 31, wherein the molecules are hydrophobic molecules.

34. The microfluidic module of claim 1, wherein the first ether-based, aliphatic polyurethane is optically clear.

35. The microfluidic module of claim 1, wherein the first ether-based, aliphatic polyurethane comprises dicyclohexylmethane-4,4′-diisocyanate, a derivative or an isomer thereof.

36. The microfluidic module of claim 1, further comprising a membrane comprising ether-based, aliphatic polyurethane.

37. The microfluidic module of claim 1, further comprising at least one cell in the at least one fluidic element.

38. A method comprising:
introducing a fluid into at least one fluidic element of a microfluidic module of claim 1, wherein at least a portion of the ether-based, aliphatic polyurethane is in contact with the fluid.

39. The method of claim 38, wherein the fluid further comprises an active agent.

40. The method of claim 38, further comprising culturing cells in the at least one fluidic element.

41. The method of claim 38, wherein the microfluidic module is connected to at least one device or instrument.

42. A method of making a multi-layered microfluidic device comprising forming a first layer comprising a fluid-contact surface of a fluidic element from at least one ether-based, aliphatic polyurethane, and bonding the first layer to a second layer.

43. The method of claim 42, wherein the first layer is bonded to the second layer by corona or plasma treatment.

44. The method of claim 42, wherein the first layer is formed by replica molding, micromachining, solid-object printing, or any combinations thereof.

45. The method of claim 42, wherein the second layer comprises ether-based, aliphatic polyurethane, glass, polydimethylsiloxane (PDMS), or any combinations thereof.

46. The method of claim 42, further subjecting the fluid-contact surface to a low temperature UV ozone treatment.

47. The method of claim 42, further culturing cells in the microfluidic device.
